(19) United States
(12) Patent Application Publication
(10) Pub. No.: US 2020/0275942 A1
(43) Pub. Date: Sep. 3, 2020

(54) DISTAL FORCE SENSING IN THREE DIMENSIONS FOR ACTUATED INSTRUMENTS: DESIGN, CALIBRATION, AND FORCE COMPUTATION

(71) Applicant: THE JOHNS HOPKINS UNIVERSITY, Baltimore, MD (US)

(72) Inventors: Iulian Ioan Iordachita, Lutherville, MD (US); Berk Gonenc, Baltimore, MD (US); Russell H. Taylor, Severna Park, MD (US); Peter L. Gehlbach, Monkton, MD (US); James T. Handa, Baltimore, MD (US)

(21) Appl. No.: 16/759,602

(22) PCT Filed: Oct. 26, 2018
(86) PCT No.: PCT/US2018/057657

§ 371 Date: Apr. 27, 2020

Related U.S. Application Data
(60) Provisional application No. 62/577,916, filed on Oct. 27, 2017.

(51) Int. Cl. A61B 17/30; A61F 9/007; A61B 34/30

(52) U.S. Cl. CPC A61B 17/30; A61B 2090/064; A61B 34/30; A61F 9/007

(57) ABSTRACT

The present invention is directed to a device to firmly grasp and manipulate delicate tissues in microsurgery, while precisely measuring tool-tissue interaction forces in three dimensions (x-y-z). The design enables precise measurement of forces at the tool tip without being influenced by other forces that may act on the tool shaft. The device of the present invention is capable of measuring axial (z) forces together with the transverse forces (x-y) on an actuated (not static) instrument. Fiber optic sensors are embedded into strategic locations of the design to decouple and precisely detect force components (x-y-z) separately. The force information is used to provide feedback to the operator, or to a robotic platform. The exerted forces on critical tissues, such as the retina in eye surgery, can be maintained at a safe level, clinical complications due to excessive forces can be lessened, safety, and outcome of microsurgical procedures can be enhanced.

### CROSS REFERENCE TO RELATED APPLICATION

This application claims the benefit of U.S. Provisional Patent Application No. 62/577,916 filed on Oct. 27, 2017, which is incorporated by reference, herein, in its entirety.

### GOVERNMENT SUPPORT

This invention was made with government support under R01EB000526 awarded by the National Institutes of Health. The government has certain rights in the invention.

### FIELD OF THE INVENTION

The present invention relates generally to surgical instruments. More particularly, the present invention relates to distal force sensing in three dimensions for actuated instruments.

### BACKGROUND OF THE INVENTION

In retinal microsurgery, membrane peeling is a standard procedure requiring the delamination of a thin (micron-scale) fibrous membrane adherent to the retina surface. After firmly grasping the membrane with a micro-forceps tool, surgeons pull the membrane away from the retina surface very slowly trying to avoid deleterious force transfer to the retina.

During the procedure, applying excessive peeling forces can harm retinal vasculature and cause serious complications potentially leading to irreversible damage and loss of vision. Most of the peeling forces were shown to be less than 7.5 mN in porcine cadaver eyes, which is well below what surgeons can feel. Therefore, continual monitoring of tool-to-tissue interaction via a sensitized instrument is essential to limit forces at a safe level either manually through auditory feedback or via robotic assistance.

Arguably the most technically demanding field of ophthalmic surgery, vitreoretinal practice has faced significant challenges due to present technical and human limitations. Epiretinal membrane surgery is the most common vitreoretinal surgery performed, over 0.5 million times annually, as reported by the Centers of Medicare and Medicaid Services. The procedure involves the dissection of a thin (micron-scale) fibrocellular tissue adherent to the inner surface of the retina, which requires first inserting the surgical tool tip to a desired depth for lifting the membrane edge without harming the underlying retina. After grasping the membrane edge using mostly a micro-forceps tool, the surgeon pulls the membrane away from the retinal surface very slowly trying to avoid deleterious force transfer to the retina. Excessive peeling forces can damage retinal vasculature and cause serious complications such as iatrogenic retinal breaks, vitreous hemorrhage and subretinal hemorrhage, leading to potentially irreversible damage and loss of vision. Prior work has found that iatrogenic retinal breaks, not related to the sclerotomy, occur in as many as 9.6%-10.7% of cases, and may result in retinal detachments in 1.7%-1.8% of cases. The problem is exacerbated by the fact that in the majority of instrument-to-tissue contact events in retinal microsurgery, the forces involved are below the tactile perception threshold of the surgeon. Among these forces, 75% were shown to be less than 7.5 mN in porcine cadaver eyes and only 19% of events with this force magnitude can be felt by surgeons. Currently, the knowledge, and hence skill, to apply appropriate peeling forces is acquired mostly through visual substitution and is qualitatively conveyed from expert surgeons to trainees. Continual quantitative monitoring of tool-tissue interaction forces via a sensitized instrument is essential to inform the operator and limit applied forces to a safe level either manually through auditory feedback or via robotic assistance. However, such a device does not exist.

Accordingly, there is a need in the art for an actuated instrument with distal force sensing in three dimensions.

### SUMMARY OF THE INVENTION

The foregoing needs are met, to a great extent, by the present invention which provides a device for surgery including a micro forceps. The device includes a guide tube having an outer wall defining an interior lumen. The interior lumen is configured to receive the micro forceps. The device includes a first force sensor positioned at a distal end of the guide tube and a second force sensor positioned at a distal end of the micro forceps. The combination of the first and second force sensors together are configured to measure tool-tissue interaction forces in three dimensions.

In accordance with an aspect of the present invention, the second force sensor is positioned axially at a center of the micro forceps. The second force sensor is configured to detect tensile, axial forces. The first force sensor is positioned laterally at the distal end of the guide tube. The first force sensor is configured to detect transverse forces at the tool tip. The first force sensor can take the form of three force sensors positioned laterally about the distal end of the guide tube. The micro forceps have a first arm and a second arm wherein first arm is straight. The second force sensor is positioned on the first arm that is straight. The second arm can include a bend. The second force sensor is positioned on the second arm that is bent.

In accordance with another embodiment of the present invention, the micro forceps have a first arm and a second arm. Both the first arm and the second arm can include a bend. The second force sensor is positioned proximal to the first and second arms of the micro forceps. The second force sensor is positioned on one of the first arm and the second arm that include a bend. The device further includes a method for calibrating the micro forceps. The device includes a motor for actuation of the device. The motor takes the form of a precision motor with an integrated encoder. An influence on the first and second sensors is modeled as a model function of a position of the motor. The model accounts for the frictional and elastic deformation forces at the micro forceps and guide tube interface inducing strain. The model accounts for strain induced on the second force sensor. The device is configured for vitreoretinal surgery. A diameter of the device is less than 0.9 mm. The calibration decouples the force readings (Fx, Fy, Fz) from the temperature and decouples the Fx, Fy, and Fz between them.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a schematic diagram of a force-sensing micro-forceps conceptual overview.

FIG. 2A illustrates an image of a fabricated prototype and a schematic diagram of an experimental setup, according to an embodiment of the present invention. FIG. 2B illustrates a graphical view of thermal drift in lateral (FBGs 1-3) and axial (FBG 4) sensor readings. FIG. 2C illustrates a linear correlation between the common mode of lateral FBGs and the axial FBG.

FIG. 3 illustrates graphical views of lateral (FBGs 1-3) and axial (FBG 4) sensor response to loading (0-25 mN) at the tool tip for three different orientations (θ=0°, 45°, 90°).

FIGS. 4A-4D illustrate graphical views of computed transverse and axial forces vs. the actual values.

FIG. 5A illustrates an image view of epiretinal membrane peeling using the motorized force-sensing micro-forceps of the present invention with the force-sensitive region of the tool inserted into the eye through a 20 Gauge sclerotomy. FIG. 5B illustrates an exploded view of components of the design, according to an embodiment of the present invention. FIG. 5C illustrates perspective and side views of a motorized actuation mechanism driving the guide tube up/down for opening/closing the jaws. FIG. 5D illustrates a schematic view of the tool coordinate frame and FBG sensor configuration.

FIG. 6A illustrates a side view of standard 23 Gauge disposable micro-forceps by Alcon Inc. (top) vs. motorized force-sensing micro-forceps, according to an embodiment of the present invention (bottom). FIG. 6B illustrates perspective and side views of a close-up view of the distal force-sensing segment, according to an embodiment of the present invention.

FIG. 7A illustrates a side view of geometric parameters of the jaw model, guide tube and the trocar attachment used in finite element simulations, according to an embodiment of the present invention. FIG. 7B illustrates a graphical view of the micro-forceps kinematics with and without the trocar attachment.

FIGS. 8A-8C illustrate graphical views of finite element simulation results showing the axial FBG response to tool actuation for various levels of friction coefficient (C_{f}) at the jaw/guide tube interface without the trocar in FIG. 8A and with the trocar attachment at the guide tube's tip in FIG. 8B.

FIG. 9 illustrates a schematic diagram of a force computation algorithm using an experimentally identified model to cancel the actuation-induced drift in FBG sensor readings based on the motor position and two distinct (linear and nonlinear) methods for transforming the corrected sensor readings into transverse (Fₓ and F_{y}) and axial (F_{z}) force information.

FIGS. 10A-10C illustrate schematic diagrams of an experimental setup, according to an embodiment of the present invention.

FIGS. 11A and 11B illustrate graphical views of the effect of opening/closing the forceps on the lateral (FBG 1,2,3) and axial (FBG 4) sensors while operating in air and in water, respectively, according to an embodiment of the present invention.

FIGS. 12A-12F illustrate graphical views of transverse and axial loading, according to an embodiment of the present invention.

FIGS. 13A and 13 B illustrate a graphical view of thermal drift in lateral and axial FBG sensor readings, respectively, during 4 test sessions each spanning a period of 225 minutes, according to an embodiment of the present invention.

FIG. 13C illustrates the Bragg wavelength shift in the axial FBG sensor shows a linear correlation with the common mode (average Bragg wavelength shift) of lateral FBG sensors with a proportionality constant of κ=0.92.

FIGS. 14A-14F illustrate graphical views of global linear calibration results for transverse forces.

FIGS. 15A and 15B illustrate graphical views of axial force (F_{z}) computation error versus the concurrent transverse load along the x-axis and the y-axis, respectively based on the global linear calibration, according to an embodiment of the present invention.

FIGS. 16A-16F illustrate graphical views of axial force computation results for local linear calibration using samples with limited roll (α<30°) and pitch (β<15°) angles, and for global nonlinear calibration.

FIGS. 17A-17I illustrate graphical views of results of local nonlinear calibration using samples with limited roll (α<30°) angles for computing Fₓ, F_{y}, and F_{Z}, according to an embodiment of the present invention.

FIGS. 18A-18I illustrate graphical views of results of the validation experiment for computing Fₓ, F_{y}, and F_{z}. FIGS. 18A-18C illustrate a comparison of computed values to the actual force level.

### DETAILED DESCRIPTION

The presently disclosed subject matter now will be described more fully hereinafter with reference to the accompanying Drawings, in which some, but not all embodiments of the inventions are shown. Like numbers refer to like elements throughout.

The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to a device that can be used to firmly grasp and manipulate delicate tissues in microsurgery, meanwhile precisely measuring tool-tissue interaction forces in three dimensions (x-y-z). The design enables precise measurement of forces at the tool tip without being influenced by other forces that may act on the tool shaft (for instance, the forces at the insertion port, if the tool is inserted through an incision to reach to the operation site, as in retinal microsurgery). The device includes fiber Bragg grating (FBG) sensors in order to sense forces at the tool tip and on the tool shaft. In addition, due to the small dimensions of the optical fibers, the diameter of the sensitized tool can be maintained relatively small, and close to the diameter of the present standard surgical instrument (for eye surgery, the tool diameter is less than 1 mm as required to fit through the scleral incision). The device of the present invention is capable of measuring axial (z) forces together with the transverse forces (x-y) on an actuated (not static) instrument. Preserving the grasping functionality of a micro-forceps, fiber optic sensors are embedded into strategic locations of the design to decouple and precisely detect each force component (x-y-z) separately. The force information can be used to provide feedback to the operator, or to a robotic platform. In this way, the exerted forces on critical tissues, such as the retina in eye surgery, can be maintained at a safe level, clinical complications due to excessive forces can be lessened, safety, and outcome of microsurgical procedures can be enhanced.

More particularly, the design of the present invention, directed to micro-forceps for grasping and manipulating tissues in microsurgery, embeds fiber-optic sensors at strategic locations to capture tool-tissue interaction forces at the instrument's tip in 3 dimensions (x-y-z). Until now, static tools (such as an ophthalmic pick) were developed to capture both transverse (x-y) and axial (z) forces at the tool tip. And actuated tools (with parts in motion for tool functionality, such as a micro-forceps) were available to detect forces only in the transverse plane (x-y). The technology developed for 3-dimensional force-sensing static tools does not directly translate to actuated tools due to the structural parts in motion (such as the grasper jaws in the case of the micro-forceps). The present invention identifies two distinct configurations for actuated instruments to detect both transverse (x-y) and axial (z) forces:
(1) Fibers can be integrated around the tubular tool shaft to capture transverse forces (x-y). The grasper jaws can be modified to have one arm flat. While the bent arm provides the grasping (open/close) functionality, the flat arm can be used to carry a force-sensitive fiber to capture the axial (z) loads without getting affected from the frictional and elastic deformation forces during open/close action of the forceps.
(2) The transverse (x-y) force sensing remains the same, but the axial (z) force sensor can be put inside the tool shaft in the center, connected to standard grasper jaws. In this configuration, the jaws need not be modified. To reduce the frictional forces associated with the actuation of the tool, an extra trocar piece is embedded at the distal end of the tubular tool shaft. This reduces the generated frictional forces as it squeezes the grasper jaws during grasping. Therefore, the problem of hysteresis can be prevented and the axial force sensor readings can be maintained accurate. In other possible embodiments the trocar and the tool shaft could form a single piece.

The developed sensor configurations allow for a small tool diameter (0.9 mm, can be reduced to 0.63 mm to match the current standard in ophthalmic surgical tools), so that it can fit through very small openings (such as the small sclera incision in eye surgery, through which the tools are inserted to reach the retinal surface). The use of optical fibers also allows for very fine force-sensitivity. Force sensing resolutions are approximately 0.25 mN in the transverse direction and less than 2 mN in the axial direction.

The actuated tool of the present invention (in contrast to static instruments) can provide firm grasping of the tissue, which may enable dexterous and comfortable tissue manipulation with less slippage. In the case of eye surgery, for a membrane peeling task for instance, this means fewer attempts taken towards the retinal surface to initiate the membrane peel, and therefore reduced risks of injuring the retina. During tissue manipulation (while peeling the membrane off the retina surface), in case excessive and dangerous tool-tissue forces are detected, the motorized design allows automatically opening the grasper jaws and quickly releasing the grasped tissue in order to avoid deleterious force transfer to critical structures and to prevent injuries. The modular design carries all the necessary actuators and sensors, which provide an operation (grasping and force sensing) independent from site of attachment. The tool can be mounted on a manual handle, or can be easily integrated and used with robotic devices for robot-assisted surgery.

A calibration method was developed to model the effect of actuation, ambient temperature changes and decouple the effect of (x-y-z) forces on the integrated sensors. For a consistent actuation effect on the sensors, the tool is motorized (rather than manual actuation by hand), so that the factors affecting sensor readings (acceleration, velocity and relative position of parts within the moving mechanism of the actuated tool) are controlled. After the effect of actuation on sensor readings is modeled as a function of the tool's state (in case of a forceps, the open/close state of jaws), by inducing various combinations of x-y-z forces on the tool tip, the effect on each sensor can be identified at that particular state, by fitting a linear or a nonlinear model. In contrast to static tools, the sensor responses may vary according to the state of the actuated tool (jaw opening of forceps). Therefore, the decoupling of x-y-z forces is by an experimental calibration that also takes into account the tool state (based on the motor position).

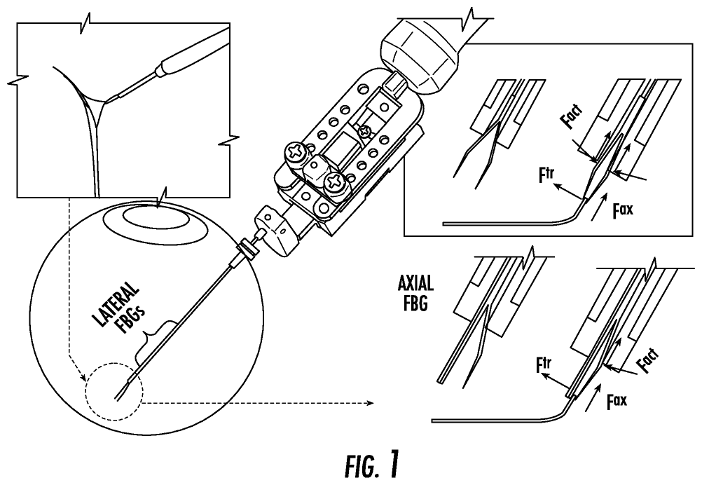

The micro-forceps of the present invention include a grasping mechanism and force-sensitive elements as shown in FIG. 1. FIG. 1 illustrates a schematic diagram of a force-sensing micro-forceps conceptual overview. (a) illustrates epiretinal membrane peeling; (b) illustrates an axial FBG in the center: actuation force (F^{act}) degrades the sensor reading; and (c) illustrates an axial FBG attached on the flattened arm of the jaw: bypassed F^{act} and direct exposure to tool tip forces (F^{ax} and F^{tr}). The actuation unit houses a compact (28×13.2×7.5 mm) and lightweight (4.5 g) piezoelectric linear motor (M3-L, New Scale Technologies Inc., Victor, N.Y.) with its embedded driver and encoder providing precise position control, which is used for opening and closing the forceps jaws. The normally-open compliant jaws are passed through a 23 Gauge guide tube and firmly anchored to the actuation unit via a set screw. The guide tube is attached to the shaft of the linear motor, so that when the motor is actuated, the guide tube is moved up and down along the tool axis, releasing or squeezing the forceps jaws.

In order to sense the applied forces after the membrane is grasped, the design employs four FBGs (Ø80 μm by Technica, Ga., USA). Three lateral FBGs are fixed evenly around the guide tube to capture the transverse forces at the tool tip. This results in a sufficiently small tool shaft diameter of 0.9 mm. The fourth FBG is responsible for detecting the tensile axial forces while the membrane is pulled away from the retina. The location of this sensor is critical to maximize accuracy. Centrally locating this sensor inside the guide tube at the distal end of the jaws of the tool could provide the best decoupling between transverse and axial forces. In this configuration, although the sensor is positioned along the neutral axis for transverse loads and thus should sense only the axial loads, preliminary experiments have shown that the frictional and elastic deformation forces generated at the guide tube/jaw interface during tool actuation significantly degrades the response of the sensor. As a remedy, an alternative concept is also presented, where the forceps jaws are modified by flattening one arm. When the guide tube is moved up and down, the flat arm is kept straight always while the other bent arm in contact with the guide tube elastically deforms to open/close the jaws. The central FBG is fixed on the flat arm of the jaws, where the sensitive region of the fiber is maintained close to the jaw tip outside the guide tube, bypassing the undesired actuation forces at the guide tube/jaw interface.

The elastic deformation of the guide tube can be modeled as an Euler-Bernoulli beam under transverse (F^{tr}) and axial (F^{ax}) loading at the tool tip, inducing a linearly proportional local elastic strain on each of the attached lateral FBGs and thus a linearly proportional shift in the Bragg wavelength of each sensor. In addition, even slight variations in ambient temperature (ΔT) may cause a drift in the Bragg wavelength. Then, the combined Bragg wavelength shift (Δλᵢ) for each lateral FBG (FBGs 1, 2 and 3) can be expressed as

Δλᵢ=Cᵢ^{F_tr}F^{tr}+C^{F_ax}F^{ax}+C^{ΔT}ΔT, where i=1,2,3 (1)

where Cᵢ^{F_tr}, C^{F_ax} and C^{ΔT} are constants. The effect of temperature and axial load in eqn. (1) can be eliminated by subtracting the common mode from the individual wavelength shift of each sensor. The transverse force is computed using the remaining differential mode (Δλᵢ^{diff}) in the linear mapping given by eqn. (2), where C^{tr} is a coefficient matrix found by calibration.

F^{tr}=C^{tr}[Δλ₁^{diff}Δλ₂^{diff}Δλ₃^{diff}]^{T} (2)

For the present invention, in addition to the elastic strain due to axial load, the axial FBG (FBG 4) also experiences a bending moment induced by the transverse force at the tool tip. Furthermore, changes in ambient temperature will induce a drift in the measured Bragg wavelength. Because the axial and transverse FBGs share the same environment, the thermal drift of the axial FBG and that of the common mode of the three lateral FBGs are linearly correlated. Based upon this hypothesis, multiplying the common mode of lateral FBGs (Δλ^{mean}) with a proper coefficient (κ) and subtracting it from Δλ₄, the effect of temperature change can be eliminated.

Δλ₄^{diff}=Δλ₄−κΔλ^{mean}=C^{ax}F^{ax}+C₄^{F_tr}F^{tr} (3)

F^{tr} is already found based upon lateral FBGs, the axial load can be computed after C^{ax} and C₄^{F_tr} constants are identified via calibration.

F^{ax}=(Δλ₄^{diff}−C₄^{F_tr}F^{tr})/C^{ax} (4)

Due to the very small dimensions and imperfections in tool fabrication, it may not be possible to accurately decouple the effect of axial and lateral loads using a linear model, especially on the axial FBG. Such a linear fitting may perform well only locally, when the transverse forces are much smaller than the axial load. In order to obtain a global estimate of force, a nonlinear fitting method based on Bernstein polynomials can be used as

$$F = {\sum }_{i = 0}^{n} {\sum }_{j = 0}^{n} {\sum }_{k = 0}^{n} {\sum }_{l = 0}^{n} {c}_{ijkl} {b}_{i , n} ( {Δλ}_{1}^{*} ) {b}_{j , n} ( {Δλ}_{2}^{*} ) {b}_{k , n} ( {Δλ}_{3}^{*} ) {b}_{l , n} ( {Δλ}_{4}^{*} ) \\ [ {F}^{tr} {F}^{ax} ] = B {C}^{Bernstein} \qquad ( 5 )$$

where the coefficients in C^{Bernstein} can be found by applying known forces (F^{tr} and F^{ax}) in various directions at the tool tip and monitoring the corresponding FBG data (Δλᵢ*).

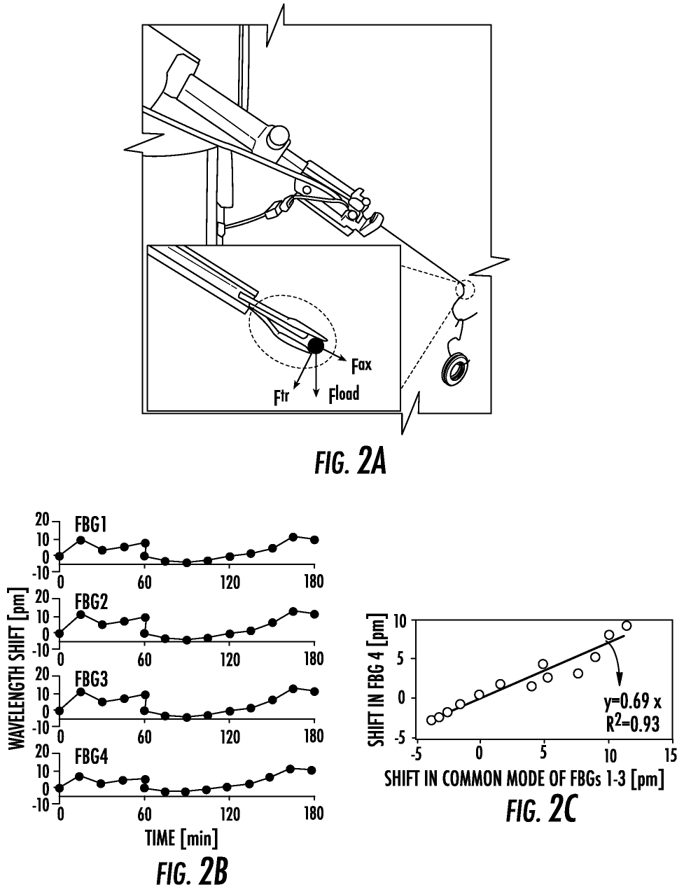

FIG. 2A illustrates an image of a fabricated prototype and a schematic diagram of an experimental setup, according to an embodiment of the present invention. FIG. 2B illustrates a graphical view of thermal drift in lateral (FBGs 1-3) and axial (FBG 4) sensor readings. FIG. 2C illustrates a linear correlation between the common mode of lateral FBGs and the axial FBG. In order to identify the constants used in the force computation algorithm, a set of calibration experiments were performed. These experimental implementations of the present invention are included to further illustrate the present invention and are not meant to be considered limiting. The goal in the first experiment was to test the hypothesis of linear correlation between the temperature drift in common mode of lateral FBGs and the axial FBG. Using an optical sensing interrogator (sm130-700 from Micron Optics Inc., Atlanta, Ga.), the Bragg wavelength of all FBGs was recorded during a 180 minute period with 15 minute intervals, while exposing the tool to routine changes in room temperature. The wavelength shift of the lateral FBGs were observed to be almost identical, as illustrated in FIG. 2B. The change in the common mode of lateral FBGs and the shift in the axial FBG could be linearly correlated as shown in FIG. 2C (R²=0.93). The corresponding proportionality constant was found to be κ=0.69.

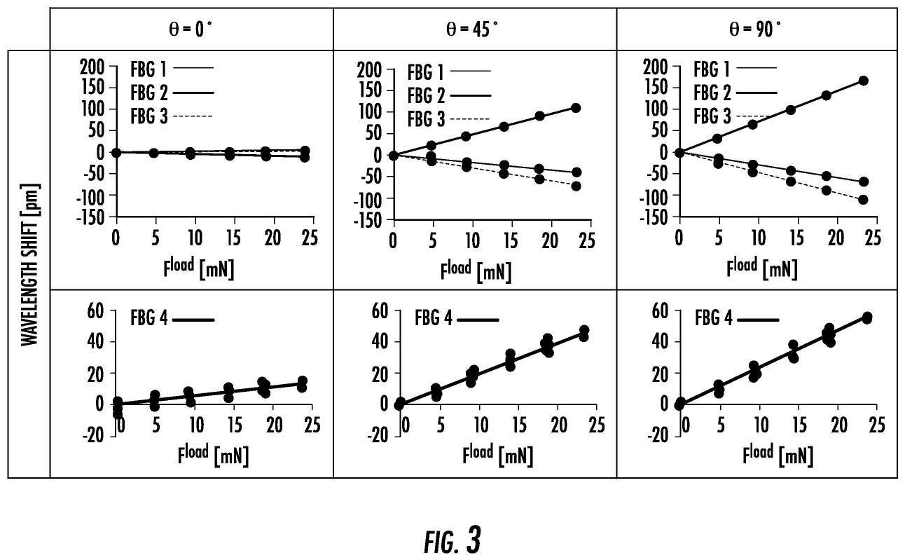

The second experiment was for modeling the lateral and axial FBG response under various forces. For this, the forceps were mounted on a rotary stage and used the jaws to grasp a wire hook, as illustrated in FIG. 2A. By hanging various loads on the hook and modulating the tool orientation (θ) the axial and transverse forces at the tool tip were changed. Measurements were taken for loads ranging from 0 to 25 mN with 5 mN intervals and at angles from θ32 0° (entirely axial loading) to θ=90° (entirely transverse loading) with 15° intervals. This produced 42 distinct loading cases; and at each orientation, the tool tip was repeatedly loaded/unloaded 3 times. The obtained response for 3 illustrative orientations is shown in FIG. 3. FIG. 3 illustrates graphical views of lateral (FBGs 1-3) and axial (FBG 4) sensor response to loading (0-25 mN) at the tool tip for three different orientations (θ=0°, 45°, 90°).

When the tool was held at θ=0°, the entire loading was axial, therefore affecting only the axial FBG linearly with a slope of 0.78. This suggests C^{ax}=0.78 pm/mN in eqn. (3). At θ=90°, the induced force was purely transverse which caused a linear response in all FBGs. The sensitivity of the axial FBG in this orientation revealed C₄^{F_tr}=2.32 pm/mN. Based upon the slope of lateral FBG response curves, the coefficient matrix in eqn. (2) was found as C^{tr}=[−0.0342 0.089 −0.0548] mN/pm. The wavelength resolution of the interrogator is 1 pm, which propagates to a transverse force resolution of 0.17 mN and an axial force resolution of 1.8 mN considering the identified coefficients for the linear method. Finally, using the entire data set of 252 measurements, the least squares problem formulated in eqn. (5) was solved to find C^{Bernstein}. The identified coefficient provided a transverse force resolution of 0.08 mN and an axial force resolution of about 1.08 mN. The fit polynomial could estimate the transverse and axial forces in the calibration data set with mean absolute residual errors of 0.11 mN and 1.23 mN and RMS errors of 0.15 mN and 1.69 mN, respectively.

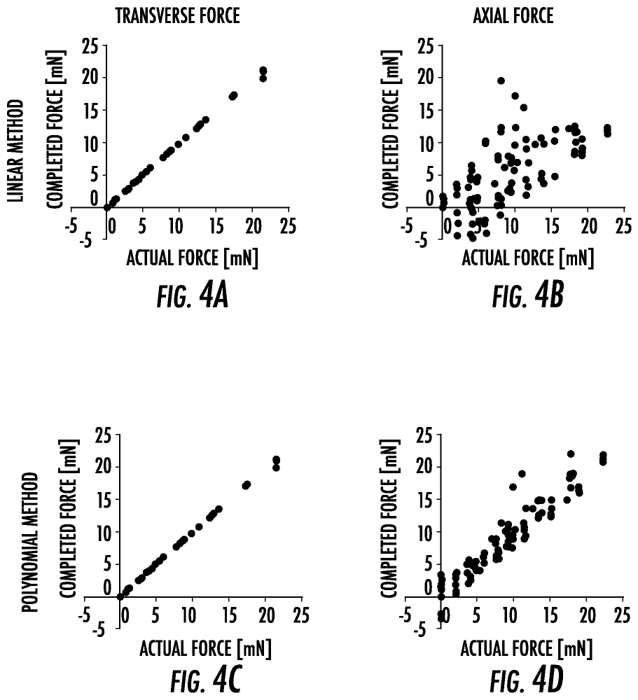

Forces ranging from 0 mN to 25 mN were applied on the tool with 5 mN intervals at 20°, 40° and 70° orientations; each test was repeated 6 times, producing 108 measurements. The data set was extended by adding 15 more measurements at randomized angles (0°-90°) and forces (0-25 mN). FIGS. 4A-4D illustrate graphical views of computed transverse and axial forces vs. the actual values. Similar accuracy for both methods in estimating the transverse load, better performance with the polynomial method for estimating the axial load.

The first attempt to estimate applied force using a linear model performed well for the transverse load with an RMS error of 0.13 mN (FIG. 4A, though did not provide an adequate accuracy in finding the axial force (FIG. 4B). The linear method produced an RMS error of 2.05 mN for cases with a transverse force less than 5 mN, but when larger (up to 25 mN) transverse loads were involved, the RMS error in the computed axial force rose up to 5.14 mN. This indicates that the linearity of sensor response is lost when large transverse forces are applied at the tool tip in addition to axial loads. The second approach, the Bernstein polynomial method, showed similar success in estimating the transverse force with an RMS error of 0.22 mN (FIG. 4C). More importantly, the accuracy in axial force estimation was significantly improved with a much smaller RMS error of 1.99 mN (FIG. 4D) for the entire force range (0-25 mN), which is closer to the required accuracy for feasibility in membrane peeling.

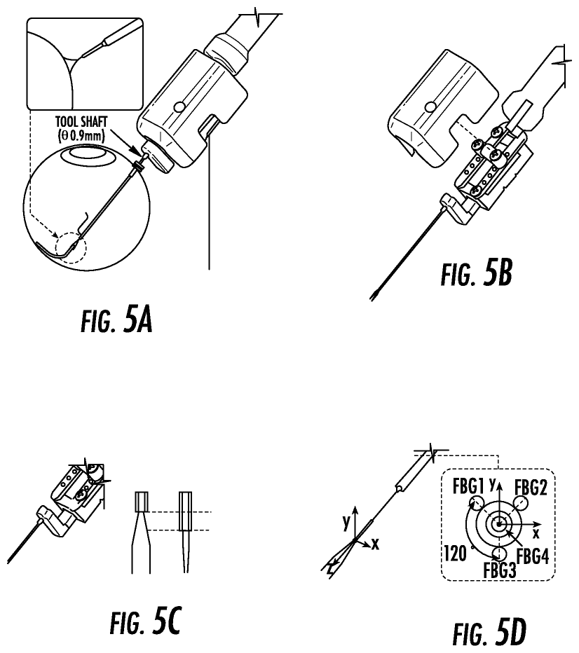

In accordance with the present invention, a force computation method was developed that (1) isolates the effect of tool actuation from the optical sensor readings (based on the motor position, therefore the tool state), (2) cancels out the drift in all sensor readings due to ambient temperature fluctuations (based on a common mode of sensor readings), and (3) computes the individual components of 3D forces at the tool tip in real time (using the model that corresponds to the current state of the tool, which was identified by an experimental calibration). The force computation methods can be linear or nonlinear. The design of the micro-forceps of the present invention involves (1) an actuation mechanism to open/close the forceps jaws for firmly grasping thin membranous layers and (2) four strategically embedded FBG sensors to measure forces about the x, y and z axes of the tool separately. The assigned coordinate system of the forceps is shown in FIGS. 5A-5D. FIG. 5A illustrates an image view of epiretinal membrane peeling using the motorized force-sensing micro-forceps of the present invention with the force-sensitive region of the tool inserted into the eye through a 20 Gauge sclerotomy. FIG. 5B illustrates an exploded view of components of the design, according to an embodiment of the present invention. FIG. 5C illustrates perspective and side views of a motorized actuation mechanism driving the guide tube up/down for opening/closing the jaws. FIG. 5D illustrates a schematic view of the tool coordinate frame and FBG sensor configuration. The axial FBG sensor (FBG 4) at the center inside the guide tube and three lateral FBG sensors integrated on the guide tube (FBGs 1-3) measure axial (F_{z}) and transverse forces (Fₓ and F_{y}) at the tool tip, respectively.

The x and y axes form the transverse plane while the z-axis lies along the tool axis. During the grasping action, the jaws elastically deform to move toward each other along the x-axis. After grasping the membrane edge, the tool is moved mostly along its z-axis and x-axis to respectively pull and peel the membrane away from the adherent inner retina surface.

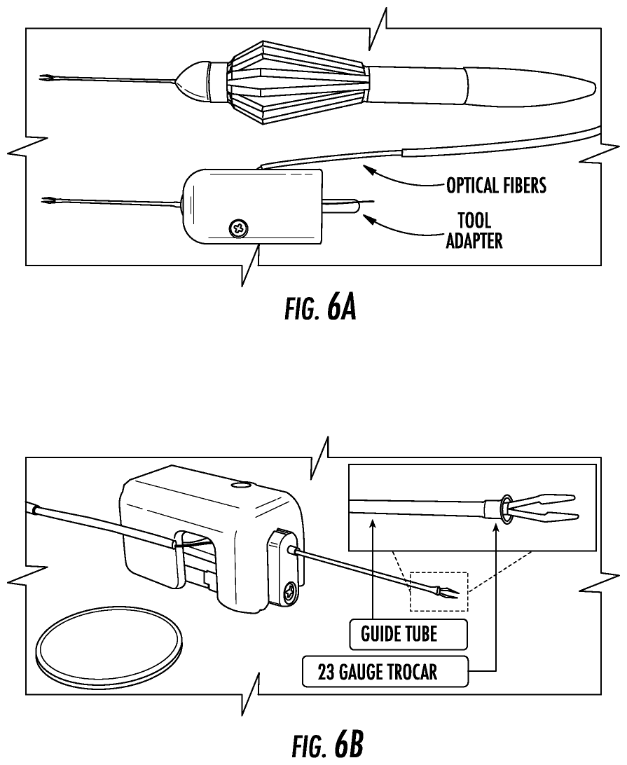

In vitreoretinal surgery, membrane peeling is often performed either by using a hook or a micro-forceps. Due to the inherent grasping capability, the latter is usually considered safer and preferred by surgeons. It enables easier and more controlled removal of the membrane from the eye with less slippage of the tissue and reduced number of grasping attempts close to the retina surface. Currently a standard tool for this procedure is the disposable micro-forceps by Alcon Inc. (Fort Worth, TX), shown in FIG. 6A. FIG. 6A illustrates a side view of standard 23 Gauge disposable micro-forceps by Alcon Inc. (top) vs. motorized force-sensing micro-forceps, according to an embodiment of the present invention (bottom). FIG. 6B illustrates perspective and side views of a close-up view of the distal force-sensing segment, according to an embodiment of the present invention. A fine-polished filleted stainless steel piece (23 Gauge trocar) was bonded at the distal end of guide tube, to modify the jaw/guide tube interface so that the reaction force during tool actuation is consistently smaller and its adverse influence on axial FBG sensor is minimal.

The Alcon device operates based on a squeezing mechanism. When the tool handle is compressed, the tubular tool shaft is pushed forward, and squeezes flexible jaws anchored to the back of the tool handle. When the tool handle is released a spring loaded mechanism pulls the tubular tool shaft back opening the jaws. Due to the moving parts within the mechanism during this actuation, studies have shown significant motion artifact at the tool tip, which limits tool tip positioning accuracy while trying to catch the membrane edge to begin delamination. Furthermore, such mechanical coupling between the tool handle and tip for actuation challenges the integration of the tool with many of the available systems for robot-assisted surgery as it can easily interfere with the operation of the attached robotic system. To address these issues, the design goal of the present invention has been toward devising a compact, lightweight and modular unit that can be controlled independently and remotely when necessary regardless of its site of attachment (such as a manual tool handle, a handheld micromanipulator or a teleoperated/cooperatively-controlled robot), resulting in the motorized micro-forceps shown in FIG. 5B.

The actuation of the micro-forceps of the present invention is provided by a compact (28×13.2×7.5 mm) and lightweight (4.5 g) piezoelectric linear motor (M3-L, New Scale Technologies Inc., Victor, N.Y.) with an embedded driver and encoder providing precise position control. The normally-open, compliant jaws are standard disposable 23 Gauge micro-forceps. They are passed through a 23 Gauge stainless steel guide tube and firmly anchored to the motor body. The guide tube is attached to the shaft of the linear motor, so that when the motor is actuated, it drives the guide tube up and down along the z-axis, releasing or squeezing (thus opening or closing) the forceps jaws, as illustrated in FIG. 5C. The parts connecting the guide tube to the motor shaft, housing the motor, anchoring the jaws to the motor body and the lid shielding the mechanism were built using 3D printed Acrylonitrile Butadiene Styrene (ABS). The assembled actuation (FIG. 6B) unit occupies a space of 1.8×1.8×3.5 mm and weighs approximately 8.9 grams, which is close to the weight of Alcon's 23 Gauge disposable micro-forceps (about 7.9 grams).

The exerted forces in membrane peeling are typically along the x-axis of the instrument during delamination and mostly tensile in z-axis while pulling the membrane away from the retina. Experiments in porcine cadaver eyes have shown forces mostly less than 7.5 mN. Measuring these very fine forces without adverse contribution from the sclerotomy requires locating the sensor inside the eye, hence a sensor that (1) can fit through a small incision (Ø≤0.9mm) on the sclera, (2) is sterilizable and biocompatible, and (3) can provide sub-mN accuracy for transverse force measurements and predict the axial load within an accuracy less than 2 mN. Based upon these constraints, the design employs 4 FBG sensors which all have one 3 mm FBG segment with center wavelength of 1545 nm (Technica S.A., Beijing, China).

Three lateral FBGs (Ø=80 μm) are fixed evenly around the 23 Gauge guide tube using medical epoxy adhesive (Loctite 4013, Henkel, Conn.) to capture the transverse forces (Fₓ and F_{y}) at the tool tip, as illustrated in FIG. 5D. New in this work is the fourth FBG sensor (Ø=125 μm) added to detect the tensile forces along the tool axis that arise when the membrane is pulled away from the retina. The location of this sensor is critical to maximize axial force sensing accuracy. As the micro-forceps is opened/closed, varying reaction forces are generated at the interface between the forceps jaws and the guide tube. The preliminary experiments (applying axial loads varying within 0-25 mN at the tool tip using the setup, which will be described in detail further herein) showed that the frictional and elastic deformation forces generated during tool actuation may significantly degrade the response of the sensor and may hinder the measurement of axial force at the tool tip. As a remedy, an alternative concept, where the forceps jaws are modified by flattening one arm and bonding the axial FBG sensor on the flat arm so that its sensitive region is located out of the guide tube close to the tip is also included. Although this architecture ensured that the axial sensor response is not affected from tool actuation, the asymmetric design complicated the calibration procedure as well as the force decoupling and computation steps. In this embodiment of the present invention, the axial FBG is maintained in the tool center inside the guide tube preserving the axial symmetry of the tool and modified the jaw/guide tube interface by attaching a fine-polished and filleted piece, the introduction section of a standard 23 Gauge trocar with the cannula section trimmed off at the distal end of the guide tube as shown in FIG. 6B. The inner diameter of the trocar fit onto the guide tube, and the flange member at its proximal end was trimmed to fit through a 20 Gauge opening (Ø=0.9 mm). Using medical epoxy adhesive (Loctite 4013, Henkel, Conn.), it was bonded onto the guide tube such that the flange member of the trocar is at the distal end of the tool and the filleted end of the trocar's hollow bore contacts the jaws for squeezing and closing them. This modification aimed at lowering the frictional forces during the actuation of the tool, preventing the jaws from getting stuck, enabling the applied axial forces to generate sensible strain on the axial FBG with the same sensitivity regardless of the opening state of the jaws. This functionality will be verified through finite element simulations in the next section, and the effect of actuation on the axial FBG response will be experimentally characterized further herein.

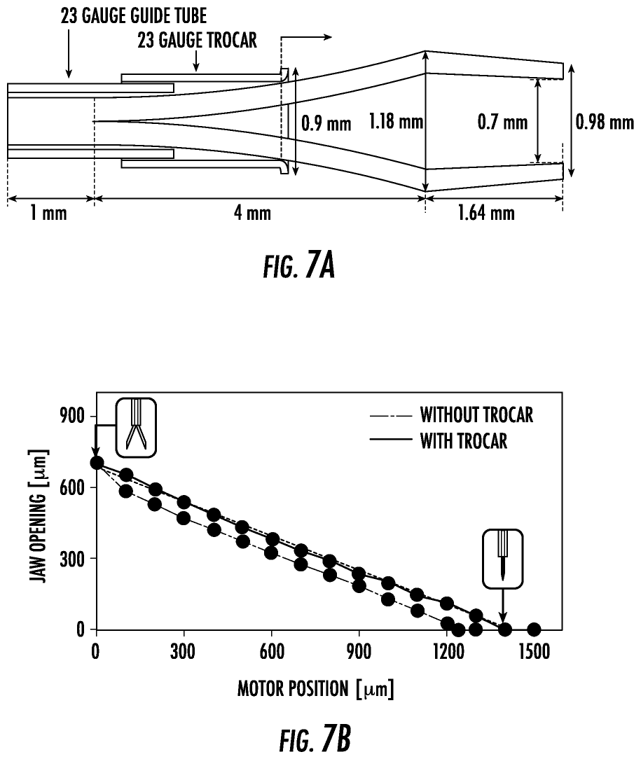

FIG. 7A illustrates a side view of geometric parameters of the jaw model, guide tube and the trocar attachment used in finite element simulations, according to an embodiment of the present invention. FIG. 7B illustrates a graphical view of the micro-forceps kinematics with and without the trocar attachment. While fully open, the jaw tips are 0.7 mm apart; full closure requires driving the motor about 1240 μm without the trocar and 1400 μm with the trocar. An almost linear response is obtained with the trocar. The motorized and encoded actuation provides a highly repeatable response, which enables a consistent correlation between the motor position and the opening between the jaws. A digital microscope is used to capture projection images of the jaws, and then used Digimizer (MedCalc Software, Belgium) software to process these images. Resulting geometric parameters are shown in FIG. 7A. Next, the identified jaw model and the 23 Gauge guide tube are implemented in ABAQUS 6.13 (Dassault Systems, USA) software to simulate the opening/closing action of jaws both with and without the trocar at the distal end of the guide tube. The material of jaws and the guide tube were both set as stainless steel, SS316 (Young's modulus=193 GPa and Poisson ratio=0.3). The jaws were maintained fixed while an increasing displacement was applied to the proximal end of the guide tube to gradually close the jaws, generating the plots shown in FIG. 7B. According to simulation results, when the jaws are fully open, the forceps tips are about 0.7 mm apart. As the guide tube is driven forward, the jaw opening decays nonlinearly at a decreasing rate without the trocar attachment. When the trocar is used, the jaws close with an almost linear response, at a rate of about 0.48 μm/μm. For full closure, using the trocar requires slightly larger translation of the guide tube, approximately 1400 μm in comparison to the 1240 μm without the trocar;

however, this is still within the travel range of the linear actuator (6 mm) and does not correspond to a significant difference in the time it takes to fully close the jaws thanks to the fast response of the actuator (>5 mm/s).

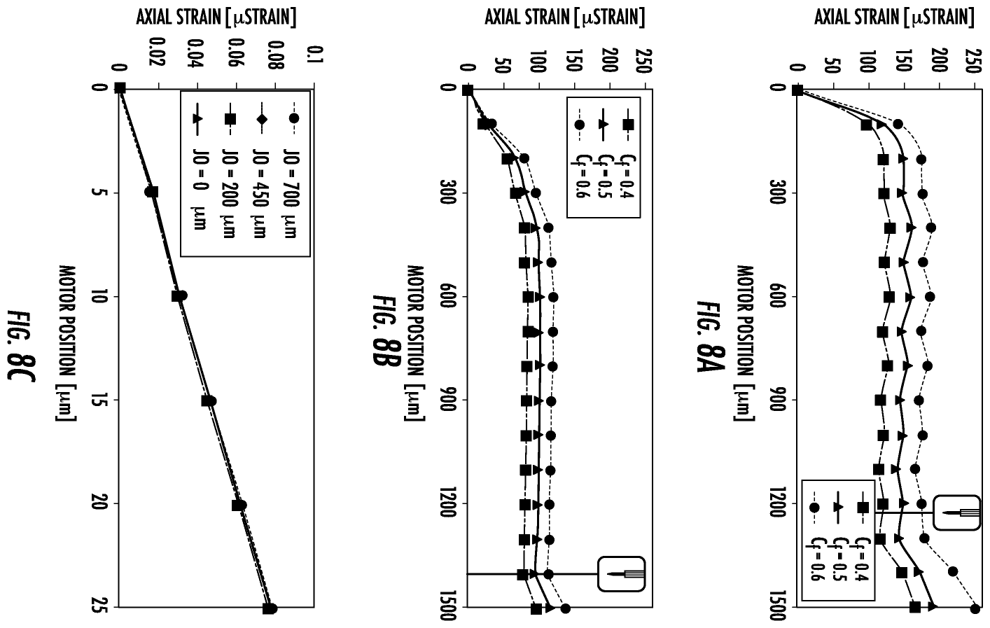

Next, the variation of the strain induced on the axial FBG during the actuation of the forceps is simulated. Since the exact value of the friction coefficient at the jaw/guide tube interface is not known, the behavior for three different coefficients (C_{f}=0.4, 0.5 and 0.6) was analyzed considering typical steel-steel dry contact properties. Results in FIG. 8A show that using the bare guide tube, the strain rises very rapidly initially at a decreasing rate during the first 300 μm of motor actuation. The trocar attachment leads to a more gradual increase in strain. After the jaws are fully closed (motor position reaches 1240 μm without the trocar and 1400 μm with the trocar), driving the motor forward further does not change the jaw opening but squeezes the jaws more, producing higher grasping force, and leading to a rapid rise in strain in all cases. Higher friction coefficients generate greater strain during actuation regardless of the trocar. However, for each value of friction coefficient, the use of trocar clearly lowers the strain level. For instance, for C_{f}=0.5, without the trocar, full closure of the forceps generates of about 140 μstrains as compared to 98.5 μstrains with the trocar. The actual effect in the Bragg wavelength of the axial sensor will be characterized experimentally herein. FIGS. 8A-8C illustrate graphical views of finite element simulation results showing the axial FBG response to tool actuation for various levels of friction coefficient (Cf) at the jaw/guide tube interface without the trocar in FIG. 8A and with the trocar attachment at the guide tube's tip in FIG. 8B. Larger friction coefficients produce more strain. Lower strain levels are observed with the trocar. FIG. 8C illustrates the force-induced strain on the axial FBG vs. the applied axial load when the trocar attachment is used: the strain is linearly correlated with the axial load, and the sensitivity is almost identical for all levels of jaw opening (JO).

In order to monitor the influence of axial forces at the tool tip, additional simulations were completed for the configuration with the trocar attachment. Forces were applied at the tip of jaws along the z-axis for various levels of jaw opening. Simulations involved forces ranging from 0 to 25 mN in increments of 5 mN. For each load condition, the motor was moved from 0 (fully open state) to 1400 μm (fully closed state), and the strain on the axial FBG sensor was recorded at each 100 μm step. After subtracting the previously identified actuation-induced strain component for each motor position (FIG. 8A), the strain was computed purely due to the applied axial load for each jaw opening. Results in FIG. 8C show that with the modified jaw/guide tube interface, the variation of force-induced strain on the axial FBG is linear for all jaw states (from fully closed to fully open); and the slope of the response remains almost the same regardless of the opening between the forceps jaws.

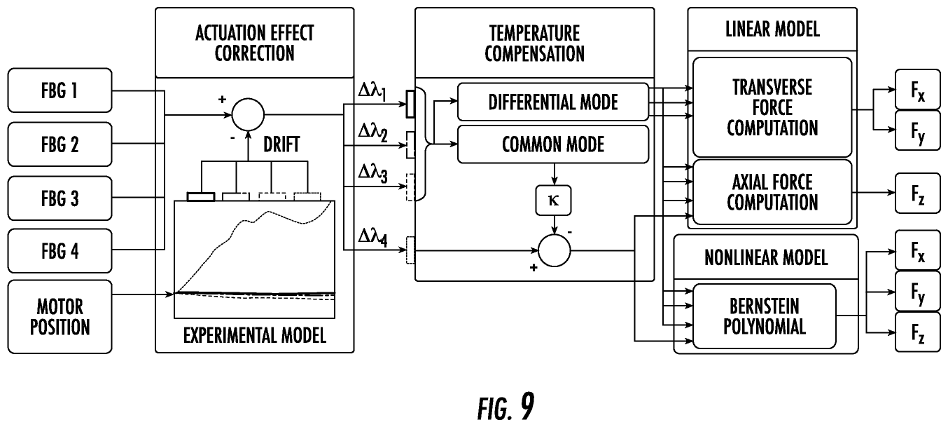

In order to transform the optical wavelength information from each embedded sensor to axial and transverse force values, the force computation algorithm summarized in FIG. 9. FIG. 9 illustrates a schematic diagram of a force computation algorithm using an experimentally identified model to cancel the actuation-induced drift in FBG sensor readings based on the motor position and two distinct (linear and nonlinear) methods for transforming the corrected sensor readings into transverse (Fₓ and F_{y}) and axial (F_{z}) force information.

As discussed above, the FBG sensors in the present invention are bonded to parts that move during the actuation of the forceps, i.e. opening/closing the jaws induces undesired drift in sensor readings. Since the actuation is performed by a precision motor with an integrated encoder, the deformation and resulting reaction forces during actuation are highly repeatable, and the influence on each FBG sensor can be modeled as a function of the motor position. This model accounts for the frictional and elastic deformation forces at the jaw/guide tube interface inducing strain especially on the axial FBG. The effect of actuation on lateral FBGs are presumably minor because the lateral FBGs are mostly sensitive to the transverse deformations, which ideally do not take place assuming perfectly aligned parts and purely linear translation of the guide tube. Furthermore, apart from the material and dimensions of the forceps structure, the model may vary depending upon the medium in which the forceps is being operated (air, water, etc.) as the coefficient of friction at the jaw/guide tube interface may change.

During the actual use of the micro-forceps, based on the identified actuation model and sensed motor position, the readings from each FBG sensor are corrected, simply by subtracting the estimated Bragg wavelength shift due to actuation. Although the temperature in patients' eyeballs is fairly constant, FBG sensors are typically very sensitive to temperature changes (approximately 10 μm/° C.). Hence, force sensing robustness against small thermal fluctuations is a desired feature. After the actuation effect correction, the drift due to thermal changes based on the common mode of lateral FBGs is cancelled out, which tool tip forces from will be further detailed in the remainder of this section. To compute the corrected and temperature compensated sensor readings, two distinct methods are described: (1) a linear method based on ideal decoupling of transverse and axial forces, (2) a nonlinear regression based on Bernstein polynomials.

Assuming small elastic deformations, the guide tube can be modeled as an Euler-Bernoulli beam under transverse (Fₓ and F_{y}) and axial (F_{z}) loading at the tool tip, inducing a linearly proportional local elastic strain on each lateral FBG and thus a linearly proportional shift in the Bragg wavelength of each sensor. In addition, even slight variations in ambient temperature (ΔT) may cause a drift in the Bragg wavelength. Then, the combined Bragg wavelength shift (Δλᵢ) for each lateral FBG sensor (FBGs 1, 2 and 3) can be expressed as

Δλᵢ=Cᵢ^{F_x}Fₓ+Cᵢ^{F_y}F_{y}+Cᵢ^{F_z}F_{z}+C^{ΔT}ΔT, i=1,2,3 (6)

where Cᵢ^{F_x}, Cᵢ^{F_y}, Cᵢ^{F_z} and Cᵢ^{ΔT} are constants associated with the x, y, z forces and the temperature change, respectively. Since the lateral FBGs are closely located within the same ambient, ideally, they are equally affected from the axial load (C₁^{F_z}=C₂^{F_z}=C₃^{F_z}=C^{F_z}) and the temperature variation (C₁^{ΔT}=C₂^{ΔT}=C₃^{ΔT}=C^{ΔT}). When the mean Bragg wavelength shift in all three lateral sensors is computed, due to axisymmetric distribution of lateral FBGs around the guide tube (120° apart from each other as shown in FIG. 5D), the terms related to the transverse forces cancel each other, resulting in the common mode (Δλ^{mean}) which is a function of the axial force and the temperature change only.

Δλ^{mean}=C^{F_z}F_{z}+C^{ΔT}ΔT (7)

The effect of temperature change and axial force in sensor readings can be eliminated by subtracting the common mode from Bragg wavelength shift of each sensor.

Δλᵢ^{diff}=Δλᵢ−Δλ^{mean}=Cᵢ^{F_x}Fₓ+Cᵢ^{F_y}F_{y}, i=1,2,3 (8)

The remaining differential mode of each sensor (Δλᵢ^{diff}) can then be used in the following equation to compute the transverse forces:

F^{tr}=[FₓF_{y}]^{T}=C^{tr}[Δλ₁^{diff}Δλ₂^{diff}Δλ₃^{diff}]^{T} (9)

where C^{tr} is a 2×3 coefficient matrix which represents the linear mapping from optical sensor readings to the force domain, and will be found via a calibration procedure.

In the design of the present invention, the axial FBG (FBG 4) lies along the tool axis ideally centered inside the guide tube, which would result in an ideal decoupling of transverse and axial loads, i.e. an axial FBG response immune to Fₓ and F_{y}, sensing purely F_{z}. However, due to the very small dimensions and imperfections resulting from tool assembly, this condition is very hard to achieve. Even if the axial FBG is slightly off-centered, besides the elastic strain due to F_{z}, the axial FBG will experience a bending moment due to Fₓ and F_{y}. In addition, excessive off-centered loading at the tool tip may also induce torsion on the axial fiber and deteriorate the FBG response, which will be negligible considering the targeted force range (0-25 mN) and the small tool diameter (0.9 mm). Furthermore, changes in ambient temperature will induce a drift in the measured Bragg wavelength. Assuming all aforementioned sources of strain contribute linearly to the axial sensor reading, the total wavelength shift observed in FBG can be formulated as

Δλ₄=C₄^{F_x}Fₓ+C₄^{F_y}F_{y}+C₄^{F_z}F_{z}+C₄^{ΔT}ΔT (10)

where C₄^{F_x}, C₄^{F_y}, C₄^{F_z} and C₄^{ΔT} are constants associated with Fₓ, F_{y}, F_{z} and temperature change, respectively. Since the axial and lateral FBGs share the same environment, the thermal drift of the axial FBG and that of the common mode of the three lateral FBGs are linearly correlated (C₄^{ΔT}=κC^{ΔT}), which will be experimentally verified herein.

Δλ₄=C₄^{F_x}Fₓ+C₄^{F_y}F_{y}+C₄^{F_z}F_{z}+κC^{ΔT}ΔT (11)

Based upon this hypothesis, by multiplying the common mode of lateral FBGs with a proper coefficient (κ) and subtracting it from Δλ₄, the effect of temperature change can be eliminated.

Δλ₄^{diff}=Δλ₄−κΔλ^{mean}=C₄^{F}^{ₓ}Fₓ+C₄^{F}^{_{y}}F_{y}+(C₄^{F_z}−κC^{F_z})F_{z} (12)

Using the linear relationship previously found for the transverse forces and rearranging the terms, the axial force can be expressed as a linear combination of each sensor's differential mode

F_{z}=C^{ax}[Δλ₁^{diff}Δλ₂^{diff}Δλ₃^{diff}Δλ₄^{diff}]^{T} (13)

where C^{ax} is a 1×4 coefficient vector which will be identified via a calibration procedure described further herein.

Due to the very small dimensions and imperfections in tool fabrication, it may not be possible to accurately decouple the effect of axial and lateral loads using a linear model, especially on the axial FBG. Such a linear fitting may perform well only locally, when the transverse forces are much smaller than the axial load, which is hard to guarantee in epiretinal membrane peeling procedure. In order to obtain a global estimate of force, a nonlinear fitting method based on Bernstein polynomials, as demonstrated earlier for a 3-DOF force-sensing pick tool, can be used:

[FₓF_{y}F_{z}]=Σⱼ₌₀ⁿΣₖ₌₀ⁿρₗ₌₀ⁿΣₘ₌₀ⁿcⱼₖₗₘb_{j,n}(Δλ*₁)b_{k,n}(Δλ*₂)b_{l,n}(Δλ*₃)b_{m,n}(Δλ*₄) (14)

where cⱼₖₗₘ denotes constant coefficients and Δλ*ᵢ denotes the differential mode (thermal drift eliminated response as described by equations (3) and (7)) of each FBG scaled down to [0,1] interval—since Bernstein polynomials exhibit good numerical stability within this range [51]—using the following equation:

$${Δλ}_{i}^{*} = \frac{{Δλ}_{i}^{duff} - {Δλ}_{i , min}^{diff}}{{Δλ}_{i , max}^{diff} - {Δλ}_{i , min}^{diff}} for i = 1 , 2 , 3 , 4 \qquad ( 15 )$$

b_{v,n}(Δλ*ᵢ) terms in equation (10) are the Bernstein basis polynomials of order n defined as follows:

$${b}_{v , n} ( {Δλ}_{i}^{*} ) = ( \begin{matrix}n \\ v\end{matrix} ) {{Δλ}_{i}^{* v} ( 1 - {Δλ}_{i}^{*} )}^{1 - v} for v = 0 … n = \\ i = 1 , 2 , 3 , 4 \qquad ( 16 )$$

In the approach of the present invention, in order to avoid overfitting with a reasonable sample size, a 2n^{d} order regression is used by setting n=2 and defining

Bⱼₖₗₘ=b_{j,2}(Δλ*₁)b_{k,2}(Δλ*₂)b_{l,2}(Δλ*₃)b_{m,2}(Δλ*₄) (17)

Then, equation (9) can be rearranged as

[FₓF_{y}F_{z}]=Σᵢ₌₀²Σⱼ₌₀²Σₖ₌₀²Σₗ₌₀²Bᵢⱼₖₗcᵢⱼₖₗ=BC^{Bernstein} (18)

where B is a 1×81 row vector formed by the product of Bernstein basis polynomials and C^{Bernstein} is a 81×3 constant matrix. The coefficients in C^{Bernstein} can be found by applying known forces (Fₓ, F_{y} and F_{z}) in various directions at the tool tip, acquiring FBG wavelength data and forming a B vector for each recorded sample, and finding the best fit in the least-squares sense.

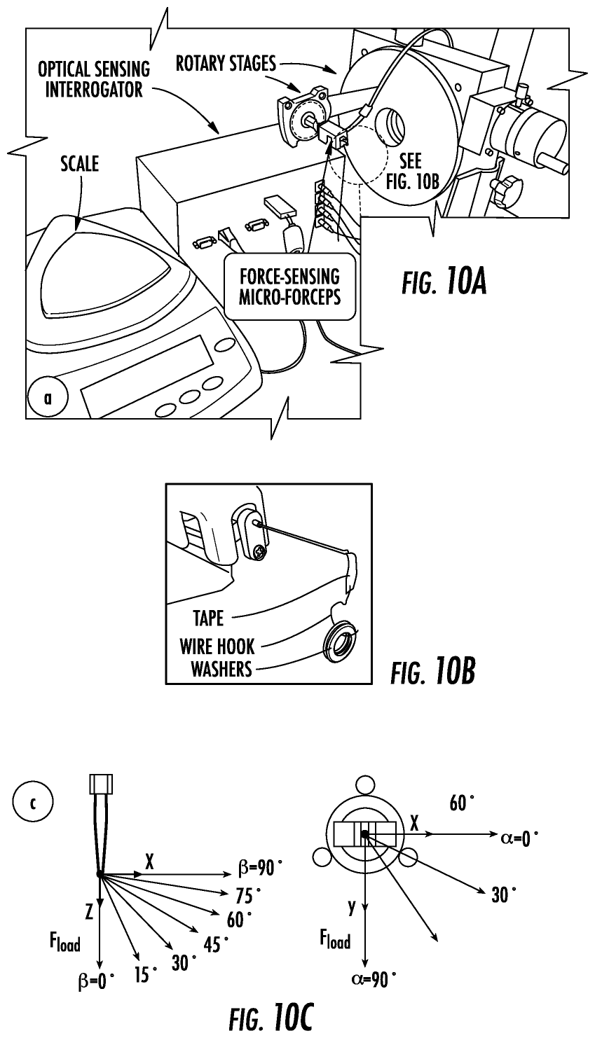

FIGS. 10A-10C illustrate schematic diagrams of an experimental setup, according to an embodiment of the present invention. FIG. 10A illustrates the 3-DOF force-sensing micro-forceps was mounted on two rotary stages to control the roll (α) and pitch (β) angles of the tool. FIG. 10B illustrates that by hanging washers onto the grasped hook, the magnitude of the applied force was changed. FIG. 10C illustrates that modulating the tool orientation (α and (β), thus the direction of the applied force, various combinations of Fₓ, F_{y} and F_{z} were applied at the tool tip.

Using the setup shown in FIG. 10A, a series of experiments were performed to model the effect of forceps actuation on force sensor readings, examine the repeatability of sensor outputs, identify calibration constants and validate the force computation methods presented herein. In order to acquire the Bragg wavelength of each FBG sensor, the setup employed an optical sensing interrogator (sm130-700 from Micron Optics Inc., Atlanta, Ga.). The force-sensing micro-forceps was mounted on a rotary stage to adjust the axial orientation (roll angle, α) of the tool. The stage was attached onto a second rotary stage to modify the pitch angle β) and tilt the tool in the vertical plane. The jaws of the forceps were closed to grasp a thin (˜80 μm thick) layer of tape carrying a wire hook. The wire hook was used to hang aluminum washers and apply varying forces at the tool tip, as illustrated in FIG. 10B. The washers were weighed by using a precision scale (Sartorius GC2502, Germany) which has a resolution of 1 mg and a repeatability of ±2 mg. The maximum test load was 23.35 mN, and each washer weighed about 4.67 mN. By changing the tool orientation (α and β) and the load hanging at the tip (Float), various combinations of Fₓ F_{y} and F_{z} were generated as shown in FIG. 10C. The resulting forces at the tool tip can be resolved into their x, y and z components using the following formulae:

Fₓ=∥F_{load}∥cos α sin β (19)

F_{y}=∥F_{load}∥sin α sin β (20)

F_{z}=∥F_{load}∥cos β (21)

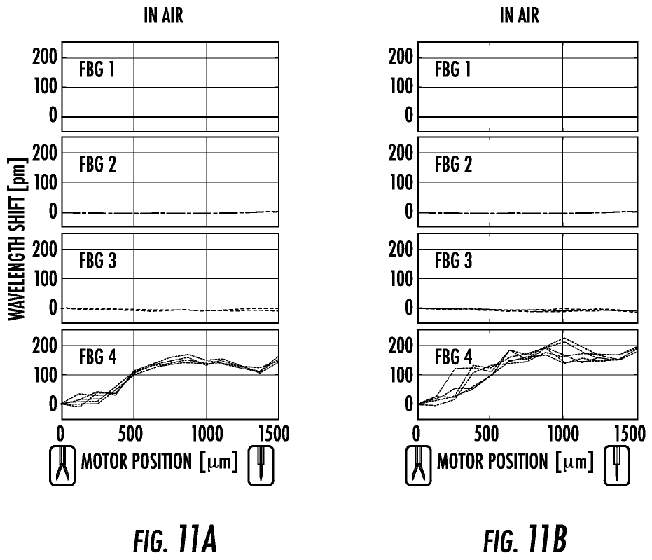

The goal of this experiment was to generate a model for compensating the detrimental effect of grasping motion on the FBG sensors. For this, the linear motor of the micro-forceps was actuated back and forth in discrete steps of 100 μm, and gradually opening/closing the forceps jaws. The jaws were fully closed after the motor was driven about 1400 μm forward from the fully open state, which is consistent with the simulation results previously presented in FIG. 11B. After each 100 μm step, the motor was stopped and the wavelength shifts of the FBG sensors were recorded. The open/close cycle was repeated 3 times, leading to 6 measurements for each sensor at each motor position. Following the identical procedure, the experiment was repeated whilst the tool tip was immersed in water.

FIGS. 11A and 11B illustrate graphical views of the effect of opening/closing the forceps on the lateral (FBG 1,2,3) and axial (FBG 4) sensors while operating in air and in water, respectively, according to an embodiment of the present invention. The actuation induces high levels of wavelength shift on the axial sensor (up to 167 pm in air and up to 222 pm in water), which exhibit a consistent variation among repeated trials, and hence can be modeled as a function of motor position for each environment.

Results in FIGS. 11A and 11B show that the motorized actuation does not induce a detectable change in the output of lateral sensors (FBGs 1, 2 and 3). However, the Bragg wavelength of the axial sensor (FBG 4) significantly varies depending on the motor position and therefore the opening state of forceps jaws in air and water. The wavelength shift profiles closely follow the axial strain variation trend that was predicted through simulations in FIG. 8B. While operating in air, wavelength shifts up to 167 pm are recorded. These recordings show much better consistency among the 6 measurements taken per each motor position in comparison to the earlier prototype without the trocar attachment. When the test is repeated in water, slightly larger shifts up to 222 pm are observed in the axial FBG output, and the consistency of readings at each motor position is relatively deteriorated. The small change can be attributed to the floating impurities inside water which can get stuck between the jaws and guide tube, and lead to larger reaction forces during actuation. Hence, the model relating wavelength shift to motor encoder readings is dependent also on the properties of the surrounding medium and needs to be experimentally tuned before each operation by opening/closing the forceps several times.

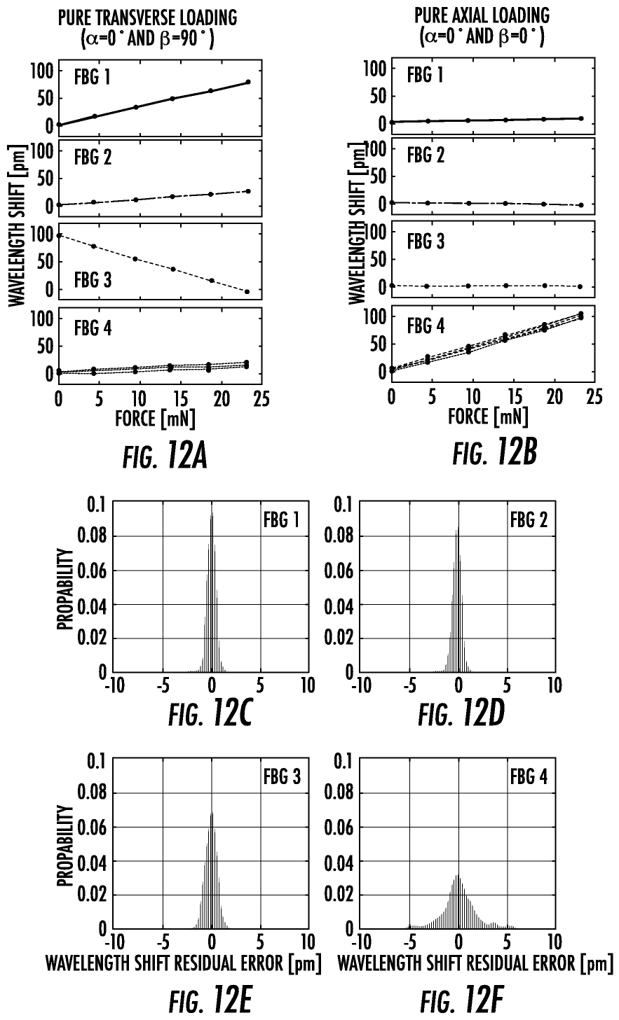

This experiment explored the consistency of the FBG sensor readings in response to axial and transverse loads. Forces were applied at the tool tip in 28 different directions by orienting the tool at 4 roll (α) and 7 pitch (β) angles. The roll angle ranged from 0° to 90° with 30° increments while the pitch angle was altered from 0° to 90° in steps of 15° . The magnitude of applied forces varied evenly at 6 levels within 0-23.35 mN. For each direction, the forcing was gradually increased up to 23.35 mN and then decremented back to zero by unloading the washers at the tool tip. The wavelength information from all four FBGs was acquired after the oscillations due to loading/unloading were fully damped out. This cycle was repeated 3 times, generating 6 measurements for each load case. For each measurement, 500 samples were recorded. As an example, FIGS. 12A and 12B show the recorded sensor response for purely transverse and axial loading conditions.

The log data involved a total of 168 distinct loading conditions, 1008 measurements and 504,000 samples. To examine the repeatability of each sensor's response, the recorded samples were grouped into 168 subsets so that each subset contained 3000 samples associated with the same loading condition. Within each subset, after identifying the mean Bragg wavelength shift for each FBG sensor, the deviations from the mean value (residuals) were computed. The residuals of all subsets were then combined to obtain the standard deviation for each FBG sensor as a measure of repeatability. FIGS. 12A-12F illustrate graphical views of transverse and axial loading, according to an embodiment of the present invention. Response of (FBG 1,2,3) and axial (FBG 4) sensors during two sample loading conditions: pure transverse loading and pure axial loading, as illustrated in FIGS. 12A and 12 B respectively. Probability distribution of Bragg wavelength shift errors for lateral, as illustrated in FIGS. 12C-12E, and axial, as illustrated in FIG. 12F. FBG sensors under 168 different combinations of transverse and axial forces. The standard deviations are less than 0.6 pm for the lateral FBGs and is about 1.96 pm for the axial FBG, indicating a highly repeatable response.

FIGS. 12C-12E show the probability distribution of the residuals for each FBG sensor. The standard deviations for the lateral FBG sensors (FBG 1, 2 and 3) are 0.47, 0.58 and 0.59 pm, respectively. The axial FBG sensor (FBG 4) exhibits slightly a more variable response with a standard deviation of 1.96 pm. The optical sensing interrogator has a wavelength repeatability of 1 pm; its wavelength stability is 2 pm typically and 5 pm at maximum. Considering these values, the FBG sensors on the tool provide reliable repeatability that is consistent with the intrinsic properties of the optical sensing interrogator.

In order to identify the coefficients used in the force computation algorithm of the present invention, a set of calibration experiments was performed. The goal in the first calibration experiment was to test the hypothesis of linear correlation between the temperature drift in common mode of lateral FBGs and the axial FBG. The Bragg wavelength variation was recorded in each FBG sensor while the tool was exposed to routine changes in room temperature, which involved gradual changes within ±2.5° C. In order to avoid disturbances due to air flow in the room, the setup was maintained inside a plastic box while acquiring data. The test was completed in 4 sessions; each session spanned a 225 minute period during which a measurement was taken in every 15 minutes. In between the sessions, the roll and pitch angles were altered to capture the effect of tool orientation on the thermal drift coefficient, if any.

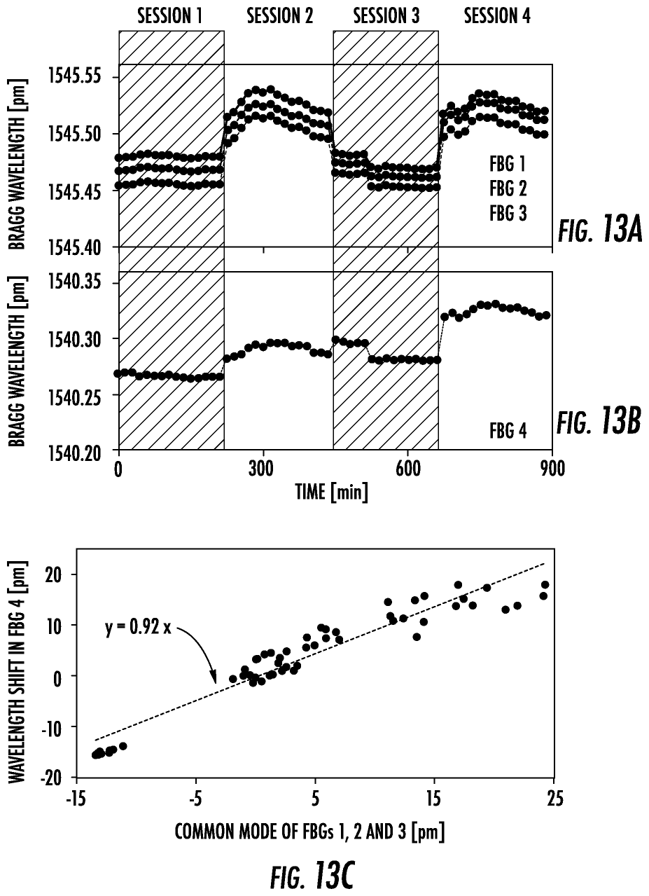

FIGS. 13A and 13B illustrate a graphical view of thermal drift in lateral and axial FBG sensor readings, respectively, during 4 test sessions each spanning a period of 225 minutes, according to an embodiment of the present invention. FIG. 13C illustrates the Bragg wavelength shift in the axial FBG sensor shows a linear correlation with the common mode (average Bragg wavelength shift) of lateral FBG sensors with a proportionality constant of κ=0.92.

The results are shown in FIGS. 13A-13C, which display the changes in Bragg wavelength of each sensor due to 2 main sources. The larger jumps while moving to the next set of measurements are due to the modified tool orientation, thus the new loading at the tool tip. The rest of the variations within each session are purely due to thermal effects. From, FIG. 13A it is observed that the lateral FBGs exhibit almost identical sensitivity to thermal changes, whereas the drift in the axial FBG was slightly smaller, as illustrated in FIG. 13B. For a quantitative comparison of thermal effects, the wavelength shift of all four sensors was recorded within each session, and computed the common mode of lateral FBGs. The wavelength shift in the axial FBG and the common mode of the lateral FBG sensors revealed a linear correlation with a covariance of 0.94, which verified that the assumptions herein were approximately correct. The corresponding proportionality constant was found to be κ=0.92, as illustrated in FIG. 13C.

The second calibration experiment was aimed at monitoring the FBG response under various combinations of transverse and axial forces. In order to collect sufficient data with a fine enough sampling grid, 504,000 samples of log data with 6 levels of forcing in 28 different directions were taken and 4 additional analyses performed: global linear calibration, local linear calibration, global nonlinear calibration, and local nonlinear calibration.

After computing the differential mode of each sensor, the linear system of equations was formed and solved by using the method of least squares. The resulting coefficient matrix for the transverse forces in equation (9) was

$${C}^{tr} = [ \begin{matrix}0.0992 & 0.0400 & - 0.1392 \\ - 0.1007 & 0.1435 & - 0.0428\end{matrix} ] mN \text{/} pm$$

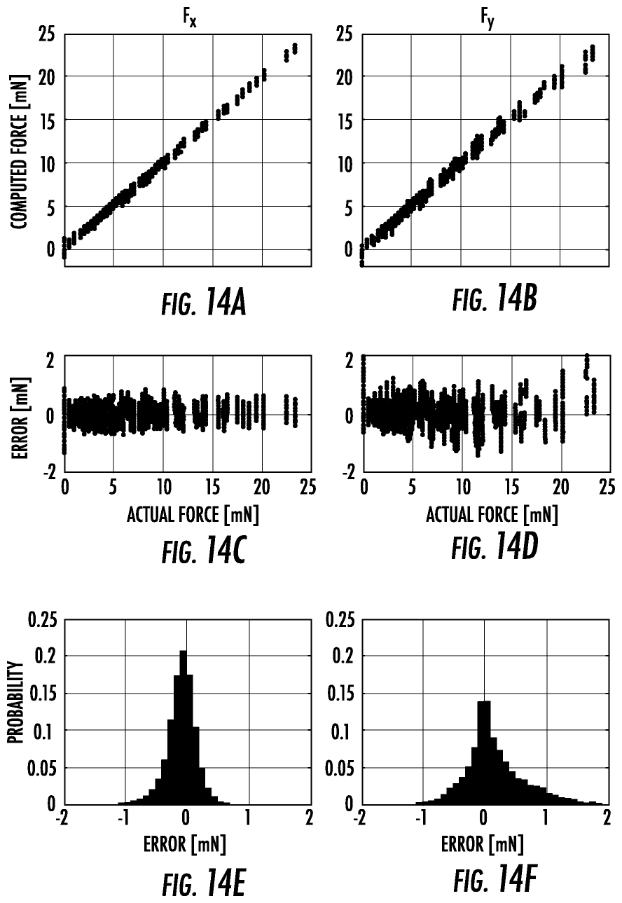

FIGS. 14A-14F illustrate graphical views of global linear calibration results for transverse forces: FIGS. 14A and 14B illustrate calculated Fₓ and F_{y} versus the actual values. FIGS. 14C and 14D illustrate residual errors versus the actual forces, FIGS. 14E and 14F illustrate a probability distribution of residuals (bin size=0.1 mN). The global linear fitting can predict the applied forces with an rms error of 0.25 mN and 0.52 mN for Fₓ and F_{y}, respectively.

Considering the wavelength resolution of the optical sensing interrogator (1 pm) and the identified coefficient matrix, the linear method produces a transverse force resolution of about 0.14 mN, which is within the initial design target of 0.25 mN. The linear fitting results are shown in FIGS. 14A and 14B for Fₓ and F_{y}, respectively. The estimated Fₓ values closely follow the actual forces with a root mean square (rms) error of 0.25 mN and a mean absolute error of 0.18 mN. A similarly accurate estimation is observed for F_{y} up to about 10 mN. However, beyond this level, slight deviations from the actual value are observed, leading to an overall rms error of 0.52 mN and a mean absolute error of 0.36 mN. The sliding contact between the jaws and the guide tube provides firm support along the x-axis of the tool, but not along the y-axis as shown in FIG. 10C. Therefore, large F_{y} forces can deform and dislocate the jaws inside the guide tube, which may change the overall geometry and deteriorate the linearity of the force sensor response. Nevertheless, this is not a major concern in membrane peeling since most of the applied forces lie along x-axis (peeling direction) due to the alignment of jaws, and excessive side loads (F_{y}) are highly unexpected. The histograms of the residual errors, as illustrated in FIGS. 14E and 14F show that the probability of errors beyond 1 mN is very low for both Fₓ and F_{y}, which have standard deviations of 0.27 mN and 0.49 mN respectively indicating a good repeatability.

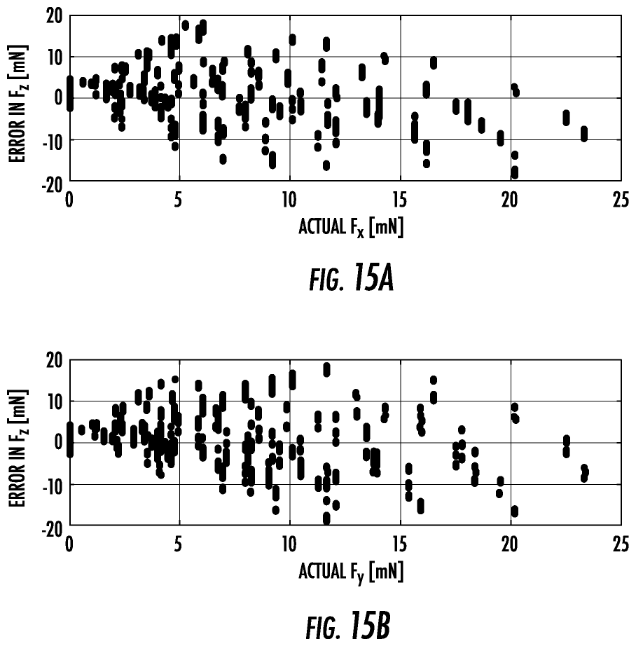

Solving the system of equations given by (13), an adequately accurate fitting was not identified to estimate the axial load. The resulting coefficient C^{ax} led to very large errors (an rms error of 8.34 mN, a mean absolute error of 6.19 mN, and a standard deviation of 3.87 mN) especially in the presence of significant transverse loads in addition to axial forces. FIGS. 15A and 15B illustrate graphical views of axial force (F_{z}) computation error versus the concurrent transverse load along the x-axis and the y-axis, respectively based on the global linear calibration, according to an embodiment of the present invention. The magnitude of errors rapidly grows when larger transverse forces are applied, deteriorating the linearity of the axial FBG. FIGS. 15A and 15B show that the magnitude of errors in axial force computation steeply rises with greater magnitude of transverse forces (both Fₓ and F_{y}), which suggests that the assumption of modeling the axial FBG response as a linear combination of axial and transverse load effects does not hold globally.

In membrane peeling, forces applied in the transverse plane are mostly along the peeling direction, which corresponds to the x-axis of the tool. Previous membrane peeling experiments on various types of artificial phantom also support that transverse loads containing large F_{y} (associated with α>30°) are not very likely in practical use of the micro-forceps. In an attempt to find a more accurate linear fitting for the axial force, a subset of the calibration data associated with α≤30° is analyzed. However, limiting a and hence F_{y} alone did not lead to any significant improvement in axial force sensing accuracy. The rms error in estimated F_{z} was still 7.08 mN and the mean absolute error was 5.41 mN. Next, a smaller subset is considered limiting both the pitch (β<15°) and roll (α≤30°) angles so that both of the transverse force components were constrained (Fₓ<6.04 mN and F_{y}<3.02 mN), and the applied forces were dominantly axial. For this subset of 72,000 samples, it was possible to obtain

$${C}^{ax} = [ 1.3216 - 0.3652 - 0.3717 \; 0.7369 ] \frac{mN}{pm}$$

which indicates an axial force resolution of about 1.32 mN.

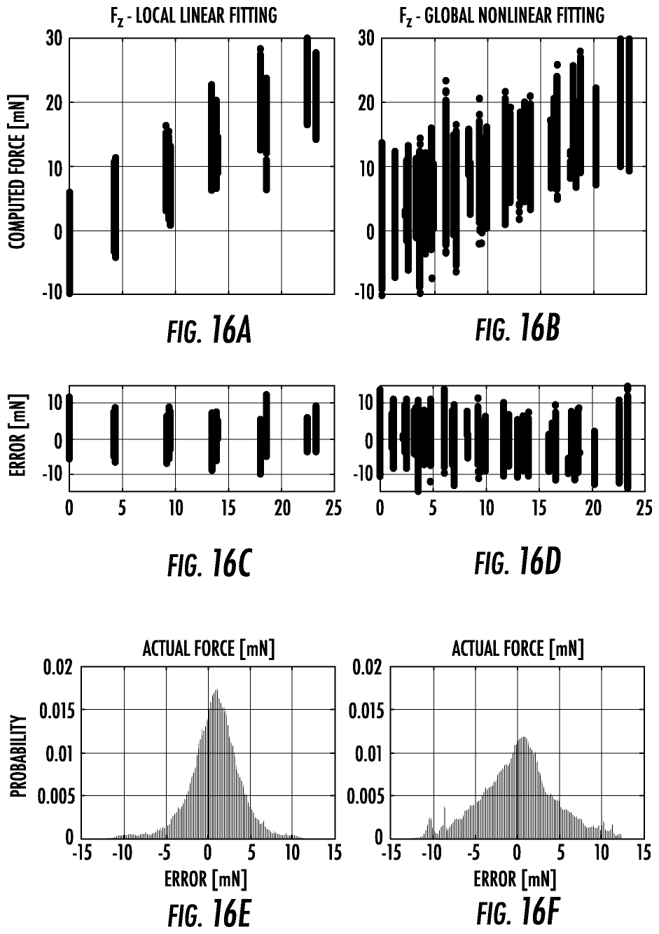

FIGS. 16A-16F illustrate graphical views of axial force computation results for local linear calibration using samples with limited roll (α≤30°) and pitch (β≤15°) angles, and for global nonlinear calibration. FIGS. 16A and 16B illustrate graphical views of the comparison of computed values to the actual force level, FIGS. 16C and 16D illustrate variation of error with respect to the axial force magnitude; FIGS. 16E and 16F illustrate probability distribution of residuals (bin size=0.1 mN). The latter provides almost the same sensing accuracy as the local fitting, but for the entire range of force directions. FIG. 16A illustrates the resulting fit, and the distribution of residuals is shown in FIGS. 16C and 16E. Accordingly, the errors are reduced to an rms value of 3.17 mN and a mean absolute value of 2.38 mN. The standard deviation is 3.09 mN, indicating slightly better repeatability. The improved accuracy with this reduced data set verifies the hypothesis on the loss of linearity in the presence of dominant transverse loads. Yet, this method is not feasible for estimating axial forces, not only because the resulting error is still over the accuracy target (2 mN), but also because in membrane peeling a significant portion of the exerted forces are transverse rather than axial, which remains in contrast to the extremely confined workspace of this method.

Using the entire log data of 504,000 samples, a nonlinear regression model based on 2^{nd} order Bernstein polynomials is fit to better estimate both the transverse and axial forces. The obtained coefficient vector C^{Bernstein} derives a resolution of 0.074 mN for Fₓ and F_{y}, and 1.85 mN for F_{z}, respectively. The accuracy in computing the transverse forces are slightly better than the linear method with an rms error of 0.15 mN for Fₓ and 0.25 mN for F_{y}. More importantly, the axial force estimation is significantly improved in comparison to the global linear fitting results. The residual error spans approximately ±4.33 mN while the mean absolute error is 3.34 mN. Although the results displayed in FIGS. 16A-16F are still unsatisfactory based upon the accuracy criterion (2 mN), they show an important improvement: the nonlinear regression provides a global axial sensing accuracy similar to what could be obtained by the linear fitting only locally.

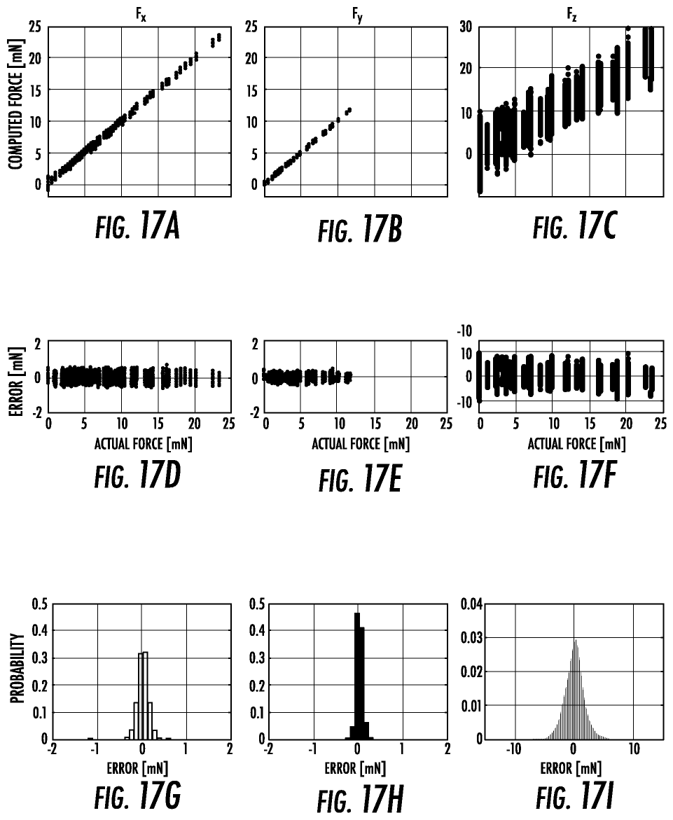

FIGS. 17A-17I illustrate graphical views of results of local nonlinear calibration using samples with limited roll (α≤30°) angles for computing Fₓ, F_{y}, and F_{z}, according to an embodiment of the present invention. FIGS. 17A-17C illustrate the comparison of computed values to the actual force level. FIGS. 17D-17F illustrate variation of residuals with respect to the force magnitude. FIGS. 17G-17I illustrate a probability distribution of residuals (bin size=0.1 mN). By limiting the roll angle (α≤30°), samples with excessive F_{y} (>11.7 mN), which are not very likely in an actual membrane peeling operation, were excluded from calibration. The rms errors in estimating Fₓ, F_{y} and F_{z} are 0.12, 0.07 and 1.76 mN respectively.

Considering that forces associated with large F_{y} forces are not very probable during an actual membrane peeling operation as discussed previously, the nonlinear calibration method was repeated using a reduced dataset (α≤30°), without limiting Fₓ (0-23.35 mN) which is expectedly the dominant force component along the peeling direction but constraining F_{y} below 11.7 mN. This corresponds to a dataset of 252,000 samples with 84 distinct loading conditions. The regression analysis revealed a coefficient vector (C^{Bernstein}) providing a finer force resolution in comparison to all of the previous fittings: 0.01 mN for Fₓ and F_{y} and 0.38 mN for F_{z}. The resulting force estimates and associated errors are plotted in FIGS. 17A-17I. The rms errors are 0.12, 0.07 and 1.76 mN for Fₓ, F_{y} and F_{z}, respectively, which are all sufficiently smaller than the initial design target (0.25 mN for transverse and 2 mN for axial forces). For the axial load, the magnitude of the residual error remains mostly within the ±5 mN envelope across the entire force range as shown in FIG. 17F. FIGS. 17G-17I illustrate the probability distributions of the residuals, which show that with the local nonlinear calibration errors mostly stay under 0.5 mN for Fₓ and F_{y}, and 5 mN for F_{z}. The standard deviations of errors are 0.12, 0.07 and 1.76 mN respectively, which show significantly better repeatability of readings in comparison to other calibration methods.

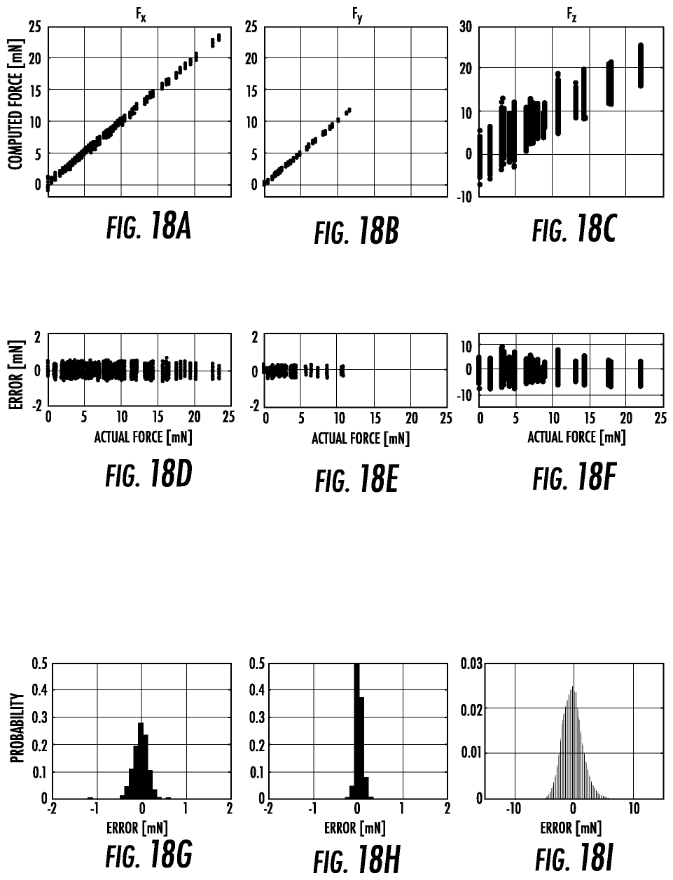

For validating the performance of the nonlinear force computation method, measurements were taken at loading conditions that were not used during the calibration, still limiting the transverse loads to the range of interest in membrane peeling, i.e. a<30° . The validation experiment consisted of forces ranging from 0 mN to 23.35 mN in increments of 4.67 mN, while holding the tool at 2 different roll angles (α=0°, 30°) and 3 different pitch angles (β=20°,40°,70°). Each case was repeated 6 times and 500 samples were collected per case. The data set was further extended by adding 15 more measurements per each roll angle at randomized pitch angles (0°-90°) and forces (0-23.35 mN), producing a total of 66 distinct loading conditions and 123,000 samples. Using a validation dataset, a similar force computation performance to what was obtained with the calibration dataset was computed. FIGS. 18A-18I illustrate graphical views of results of the validation experiment for computing Fₓ, F_{y}, and F_{z}. FIGS. 18A-18C illustrate a comparison of computed values to the actual force level. FIGS. 178D-18F illustrate variation of residuals with respect to the force magnitude. FIGS. 18G-18I illustrate a probability distribution of residuals (bin size 32 0.1 mN). Tested data consists of loading conditions that were not involved during calibration. The identified local nonlinear regression can still accurately predict the applied forces with rms errors of 0.16, 0.07 and 1.68 mN for Fₓ, F_{y} and F_{z}, respectively.

The locally fit nonlinear model is able to accurately predict the applied transverse forces within the considered force range, 0-25 mN for Fₓ and 0-11.7 mN for F_{y}. The rms errors are 0.16 mN and 0.07 mN for Fₓ and F_{y}, respectively. The axial forces are captured with an rms error of 1.68 mN, which is satisfactorily smaller than the design target of 2 mN. The standard deviation of errors indicate a force sensing repeatability of 0.15 mN, 0.07 mN and 1.67 mN about x, y and z axes, respectively. These results demonstrate that the 3-DOF force-sensing micro-forceps with the nonlinear force computation method can provide measurements within the desired sensitivity and accuracy.

The earlier works demonstrated that the temperature compensation method described herein provides robust transverse and axial force measurements against thermal changes for other tools. The first set of the calibration experiments explored the thermal influence on each FBG sensor output in response to slow and gradual ambient temperature variation. In case of sudden ambient temperature variations though, such as the instant when the tool is inserted into the eye, whether the linear correlation between the axial sensor response and the common mode of lateral sensors is valid remains controversial. In practice, this issue can be alleviated by rebiasing the force sensor to adapt to the new temperature level right after the tool is placed inside the eyeball. After this time, the expected thermal fluctuations inside the eye will be relatively small and gradual so that the thermal drift method cancelling based on common mode of lateral sensors can be used. There are several potential solutions that can improve the robustness of the force-sensing tool to thermal changes: adding a separate reference FBG, using two different wavelengths, or using different optical modes.

In order to compute forces from the optical sensor information, the use of a nonlinear fitting based on second order Bernstein polynomials was explored. Increasing the polynomial order may potentially improve the sensing accuracy, especially in the axial direction. However, identifying a higher order polynomial without overfitting requires calibration experiments that capture the FBG sensor outputs for a finer grid of forces. Such extensive dataset is quite challenging to acquire with the presented setup. Furthermore, the manual operation of rotary stages and loading/unloading of washers to modulate the loading induced at the tool tip is prone to human error. However, using a robotic calibration approach similar to, it is possible to collect more samples reliably in a shorter time and identify a more accurate higher order nonlinear force computation model.

In the calibration and validation experiments, the applied forces in the axial direction were always tensile. Therefore, the identified models do not describe the behavior for compressive loads. In addition, the final accuracy and resolution values were obtained for a limited force range of 0-25 mN with minor force component perpendicular to the peeling direction (F_{y}<11.7 mN). Although, these may be interpreted as limitations of the approach, they are highly relevant to the actual clinical scenario. In epiretinal membrane peeling, to avoid retinal injuries, the magnitude of forces need to be maintained typically below 10 mN. Also, the membrane is pulled away from the retina surface, which causes axial loads on the micro-forceps tip to be tensile if any. The exerted forces in the transverse plane follow the direction of tool motion, which means they are usually along the opening/closing direction of the jaws (x-axis of the tool). These practical facts support the constraints of the force computation model for epiretinal membrane peeling. Nevertheless, using the same experimental method, the instrument can be calibrated to the desired force domain for a different application as well.

Based on the practically relevant force ranges, using the nonlinear method showed that the rms error in axial force sensing could be lowered under 2 mN. This is a significant improvement on the previously reported results, and is presumably useful for limiting intra-operative forces and preventing retinal injuries in epiretinal membrane peeling. Nevertheless, for other aims—including quantitative assessment of differing surgical techniques, objective evaluation of the surgical performance and accurate modeling of retinal tissues—future work aims to further improve the axial sensing accuracy. Some potential methods include using higher order nonlinear models for force computation, and exploring customized sensor architectures that provide better decoupling between axial and transverse forces.

The present invention includes a novel force-sensing micro-forceps that can capture 3-DOF tool-tissue interaction forces during membrane peeling in vitreoretinal surgery. This is the first micro-forceps that can sense not only transverse but also the axial forces at the tool tip to be used in vitreoretinal surgery. Main contributions are the calibration procedure and force computation methodology using FBG sensor readings influenced by a mixture of sources, such as thermal changes and tool actuation apart from tool tip forces. In design, the sensitized segment of the instrument was located close to tool apex inside of the eye so that tool-tissue interaction forces at the tool tip could be detected without the influence of any other forces along the tool shaft. By strategically embedding 4 FBG sensors on the tool shaft, the decoupling between transverse and axial forces was maximized. The grasping functionality was provided via a compact motorized unit which enabled tool actuation without requiring any mechanically coupled handle mechanism in contrast to the existing standard micro-forceps and ensured a highly repeatable behavior in FBG sensor outputs during actuation. Through experiments inside air and water, the actuation influence on sensor outputs was determined as a function of motor position. The resulting model was later used to cancel out the undesired influence on the sensors due to tool actuation. Experiments were carried out to test the repeatability of sensor outputs, calibrate the force sensor and validate its performance. For computation of forces, two distinct methods were explored: a linear regression and a nonlinear fitting based on second-order Bernstein polynomials. Results showed that the FBG sensors provide a highly repeatable output, and the nonlinear force computation approach provides superior accuracy. Based upon the developed calibration and force computation methods, future studies aim at optimization of the tool structure and fabrication process to improve the force sensing accuracy.

This work modeled and evaluated the force response of the tool of the present invention based upon static measurements, where samples were acquired after the response of each sensor reached steady-state. The dynamic response of the tool and its performance in estimating rapidly changing force profiles will be explored in future experiments. In addition, the micro-forceps was devised as a modular unit so that it can be easily combined with robotic systems. Future work aims at integrating the tool with a robotic assistant by combining motion compensation and image-guidance features with various force feedback and force control methods to aid safe grasping and peeling of epiretinal membranes. Upon system integration, feasibility studies will be performed initially on artificial membrane peeling phantoms, then on biological membranes, and eventually using ex vivo and in vivo animal models.

The control of the present invention can be carried out using a computer, non-transitory computer readable medium, or alternately a computing device or non-transitory computer readable medium incorporated into the robotic device.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computer designed specifically for this purpose. The computing device can be unique to the present invention and designed specifically to carry out the method of the present invention. The operating console for the device is a non-generic computer specifically designed by the manufacturer. It is not a standard business or personal computer that can be purchased at a local store. Additionally, the console computer can carry out communications through the execution of proprietary custom built software that is designed and written by the manufacturer for the computer hardware to specifically operate the hardware.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention. While exemplary embodiments are provided herein, these examples are not meant to be considered limiting. The examples are provided merely as a way to illustrate the present invention. Any suitable implementation of the present invention known to or conceivable by one of skill in the art could also be used.

1. A device for micro surgery comprising:
micro forceps;
a guide tube having an outer wall defining an interior lumen, wherein the interior lumen is configured to receive the micro forceps;
a first force sensor positioned at a distal end of the guide tube; and
a second force sensor positioned at a distal end of the micro forceps;
wherein the combination of the first and second force sensors together are configured to measure tool-tissue interaction forces in three dimensions.

2. The device of claim 1 further comprising the second force sensor being positioned axially at a center of the micro forceps and wherein the second force sensor is configured to detect tensile, axial forces.

3. The device of claim 1 further comprising the first force sensor being positioned laterally at the distal end of the guide tube and wherein the first force sensor is configured to detect transverse forces at a tip of the micro forceps.

4. The device of claim 3 wherein the first force sensor comprises three force sensors positioned laterally about the distal end of the guide tube.

5. The device of claim 1 further comprising the micro forceps having a first arm and a second arm wherein first arm is straight.

6. The device of claim 5 wherein the second force sensor is positioned on the first arm that is straight.

7. The device of claim 5 wherein the second arm comprises a bend.

8. The device of claim 7 wherein the second force sensor is positioned on the second arm that has a bend.

9. The device of claim 1 further comprising the micro forceps having a first arm and a second arm wherein both the first arm and the second arm comprise a bend.

10. The device of claim 9 wherein the second force sensor is positioned proximal to the first and second arms of the micro forceps.

11. The device of claim 9 wherein the second force sensor is positioned on one of the first arm and the second arm that comprise a bend.

12. The device of claim 1 further comprising a method for calibrating the micro forceps.

13. The device of claim 1 further comprising a motor for actuation of the device.

14. The device of claim 13 wherein the motor takes the form of a precision motor with an integrated encoder.

15. The device of claim 13 wherein an influence on the first and second sensors is modeled as a model function of a position of the motor.

16. The device of claim 15 wherein the model accounts for the frictional and elastic deformation forces at the micro forceps and guide tube interface inducing strain.

17. The device of claim 16 wherein the model accounts for strain induced on the second force sensor.

18. The device of claim 1 wherein the device is configured for vitreoretinal surgery.

19. The device of claim 19 wherein a diameter of the device is less than 0.9 mm.

20. The device of claim 12 wherein the calibration decouples the force readings (Fx, Fy, Fz) from the temperature and decouples the Fx, Fy, and Fz between them.
